(19) United States
(12) Patent Application Publication
(10) Pub. No.: US 2019/0321208 A1
(43) Pub. Date: Oct. 24, 2019

(54) CAPSULE TO BE INGESTED BY A SUBJECT

(71) Applicant: MELCAP SYSTEM LTD., Haifa (IL)

(72) Inventors: Boris Voznesensky, Raanana (IL); Alexander Melamud, Haifa (IL); Michael Taborisky, Migdal HaEmeq (IL); Rita Zucker, Haifa (IL)

(21) Appl. No.: 15/959,306

(22) Filed: Apr. 23, 2018

(51) Int. Cl. A61F 5/00

(52) U.S. Cl. CPC A61F 5/0036; A61B 5/07

(57) ABSTRACT

A capsule to be ingested by a subject, the capsule may include an outer enclosure configured to dissolve in gastric juice; a secondary enclosure that is permeable for gastric juice, is at least partially surrounded by the outer enclosure when in a folded state, and has a fully expanded volume; and an expandable polymer element that is surrounded by the secondary enclosure; wherein a fully expanded volume of the expandable polymer element exceeds the fully expanded volume of the secondary enclosure.

### BACKGROUND

Obesity is a major health problem in developed countries. In the United States, the complications of obesity affect nearly one in five individuals at an annual cost of approximately $40 billion. Except for rare pathological conditions, weight gain is often directly correlated to overeating.

One strategy for controlling the individual's food intake is via the use of intragastric volume occupying devices. Such devices are placed in the stomach and occupy a portion of its interior.

Properly placed and sized, such intragastric devices provide the patient with a feeling of satiety after ingesting only a small amount of food. Typically, the individual's caloric intake is thus diminished due to the subjective feeling of fullness. There are a number of available volume-occupying devices. Many must be introduced and removed using laparoscopic or other complex gastric procedures.

Intragastric balloons have been in clinical use for several years. Their success in the treatment of certain individuals with morbid obesity is well accepted.

Although diets, diet pills, and other weight-reducing plans may be effective for a short period of time, studies have shown that over 96 percent of those who lose as much as 100 pounds, tend to regain that lost weight, plus additional pounds, within 3 years. Known methods to successfully treat the morbidly obese are invasive and permanent. For example, surgical procedures such as gastric bypass or vertical banded gastroplasty have been used to reduce the size of the stomach or decrease absorption in the intestines of severely obese patients. However, these surgeries carry significant risks inherent in all invasive surgeries, and they cannot be modified or reversed.

More recently, implantable systems and methods have been developed for reversibly treating obesity by stimulating the vagus nerve to produce a feeling of satiety, which leads to weight reduction. Side effects associated with implantable systems include risks associated with surgery, including bleeding, infection, and pain. Other problems associated with the implantation of an implanted nerve stimulation device is neurostimulator migration leading to secondary surgery, pain or seroma at site of the neurostimulator, perforation of the stomach wall, migration of leads, and allergenic or immune system response to implanted materials. Implantable vagus nerve stimulation systems have also been used to control seizures in patients.

It is generally known that the rate at which an oral controlled drug delivery system delivers the drug into the gastrointestinal fluids may not the same as the rate at which it releases the drug into a test aqueous fluid, because the gastrointestinal fluid's pH and composition change with the specific location of the drug delivery system in the gastrointestinal tract i.e. from the stomach to the colon, fasted versus fed state, type and amount of food ingested, and also due to variations in these factors from individual to individual. In addition, the drug may not be absorbed in the same manner and propensity as it moves from the stomach to the colon. Some drugs have an “absorption window” i.e. they are absorbed only from the upper parts of the gastrointestinal tract, whereas there are others whose absorption from the colon is not uniform or complete. Thus, the location of the controlled drug delivery system in the gastrointestinal tract, as well as the rate at which the controlled drug delivery system moves from the stomach to the colon, represent important factors that need to be considered in the design of an oral controlled drug delivery system. It is thus known to those skilled in the art that an oral controlled delivery system should be designed not only with control over the rate at which it releases the drug over the drug delivery time period (temporal control), but also control over the location from which it is delivered (spatial control). Such spatial control can be achieved by prolonging the period of retention of the system in the stomach. Gastric retention systems are also beneficial when the drug is effective locally in the stomach. Drugs absorbed in the upper part of the gastrointestinal tract may exhibit variability in absorption due to inter and intra-individual variability in gastric emptying and gastrointestinal motility. This variation in absorption is addressed partly by a gastric retention drug delivery system and may be further addressed by administering a dosage form comprising the drug, such that a part of the drug is available as immediate release, and a part is available as sustained or controlled release.

### SUMMARY

There may be provided a capsule to be ingested by a subject, the capsule may include an outer enclosure configured to dissolve in gastric juice; a secondary enclosure that may be permeable for gastric juice, may be at least partially surrounded by the outer enclosure when in a folded state, and has a fully expanded volume; and an expandable polymer element that may be surrounded by the secondary enclosure; wherein a fully expanded volume of the expandable polymer element exceeds the fully expanded volume of the secondary enclosure.

The secondary enclosure may be configured to at least partially self-disintegrate after a first period of time in gastric juice.

The secondary enclosure may be configured to withstand gastric juice until exiting the stomach.

The expandable polymer element may be configured to be formed from at least one out of dry granules, pellets, powders, micro tablets, mini tablets, and compressed element.

The secondary enclosure may include multiple apertures with shapes and sizes that prevents exit of the at least one of the dry granules, pellets, powders, micro tablets, mini tablets, and compressed element.

The secondary enclosure may be hydrophilic.

The secondary enclosure may include at least two sections.

The secondary enclosure, when in an expanded state, may be configured to be stuck in an antrum region of the subject until an expiration of the first period of time.

The capsule may include secondary enclosures that may be at least partially surrounded by the outer enclosure when in a folded state.

The secondary enclosures may be connected to each other by at least one connector that may be configured to at least partially self-disintegrate after a second period of time in gastric juice.

The secondary enclosures, while in an expanded state and while connected to each other may form a structure that may be stuck in the stomach.

The secondary enclosures, while in an expanded state and after disconnecting from each other, may be small enough to individually exit through the pylorus.

The first secondary enclosure may surround a first set of expandable polymer elements having a first swelling ratio and a first expansion profile; and wherein a second enclosure may surround a second set of expandable polymer elements having a second swelling ratio and a second expansion profile; and wherein the first swelling ratio may be smaller than the second swelling ratio.

The first expansion profile may exhibit a higher expansion rate than the second expansion profile.

The capsule may include a housing that may surround an electronic block.

The housing may include electrodes, and the electronic block may include a power source, a control unit with a wireless communication module and a generator.

The capsule may include a temperature sensor

The housing may be hermetically sealed.

The housing may be partially transparent.

The capsule may include a camera that may be configured to obtain images through the housing.

The capsule may include a light source.

The capsule may include a pH sensor.

The capsule may include a drug storage and a third secondary enclosure that may surround the drug storage.

The drug storage may store a drug in a slow—release packaging having a pre-determined period of self-disintegration.

The third secondary enclosure may include an electronic block.

The electronic block may include a power source, a control unit and a drug delivery system.

The drug delivery system may be a drug delivery pump.

The capsule may include a control switch and an internal power supply unit; wherein the control switch, once fed with a control signal from a capsule activator, may be configured to activate the internal power supply unit.

The control switch may be coupled to electrodes of the capsule.

The capsule may include a control unit, a first electrode, a second electrode, a control switch that has a first input and a second input, and an internal power supply; wherein the first input may be coupled to the first electrode; wherein the second input may be coupled to the control unit; wherein the control switch may be coupled to the internal power supply; wherein the control switch, once fed with a voltage from an external power supply may be configured to activate the internal power supply.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates an example of a capsule;

FIG. 2 illustrates an example of a capsule;

FIG. 3 illustrates an example of a capsule;

FIG. 4 illustrates an example of a capsule;

FIG. 5 illustrates an example of a capsule;

FIG. 6 illustrates an example of a capsule;

FIG. 7 illustrates an example of a capsule;

FIG. 8 illustrates an example of a capsule;

FIG. 9 illustrates an example of a capsule;

FIG. 10 illustrates an example of a capsule;

FIG. 11 illustrates an example of a capsule;

FIG. 12 illustrates an example of a capsule;

FIG. 13 illustrates an example of a capsule;

FIG. 14 illustrates an example of a capsule and an activator;

FIG. 15 illustrates an example of a capsule and an activator;

FIG. 16 illustrates an example of a capsule; and

FIG. 17 illustrates an example of a capsule.

### DETAILED DESCRIPTION OF EMBODIMENTS

Any reference to a capsule should be applied, mutatis mutandis to a method that is executed by a capsule.

Any reference to method should be applied, mutatis mutandis to a capsule that is configured to execute the method.

The term “and/or” is additionally or alternatively.

There may be provided various capsules include a secondary enclosure that surrounds expandable polymer elements. The aggregate volume of the expandable polymer elements when fully expanded exceed the volume of the fully expanded secondary enclosure.

During a certain period of time, when located inside a subject—especially when located within the stomach of the subject—the secondary enclosure expands to a fully expanded state and the expandable polymer elements expand to a partial expanded state—as the secondary enclosure (even when fully expanded) prevents the expandable polymer elements to reach their fully expanded state.

When in the partially expanded state the expandable polymer elements apply a force against the secondary enclosure—thus forming a structure that is rigid and/or uncompressed.

The rigidness of the structure allows the structure to stay in the stomach even when pressure is applied on the structure.

The structure may be big enough (length, width and depth) to remain in the stomach—and not exit through the pylorus. For example—at least two dimensions out of the length, width and depth of the structure may exceed the depth and width of the pylorus antrum. Each dimension of the at least two dimensions may exceed—for example—30 mm.

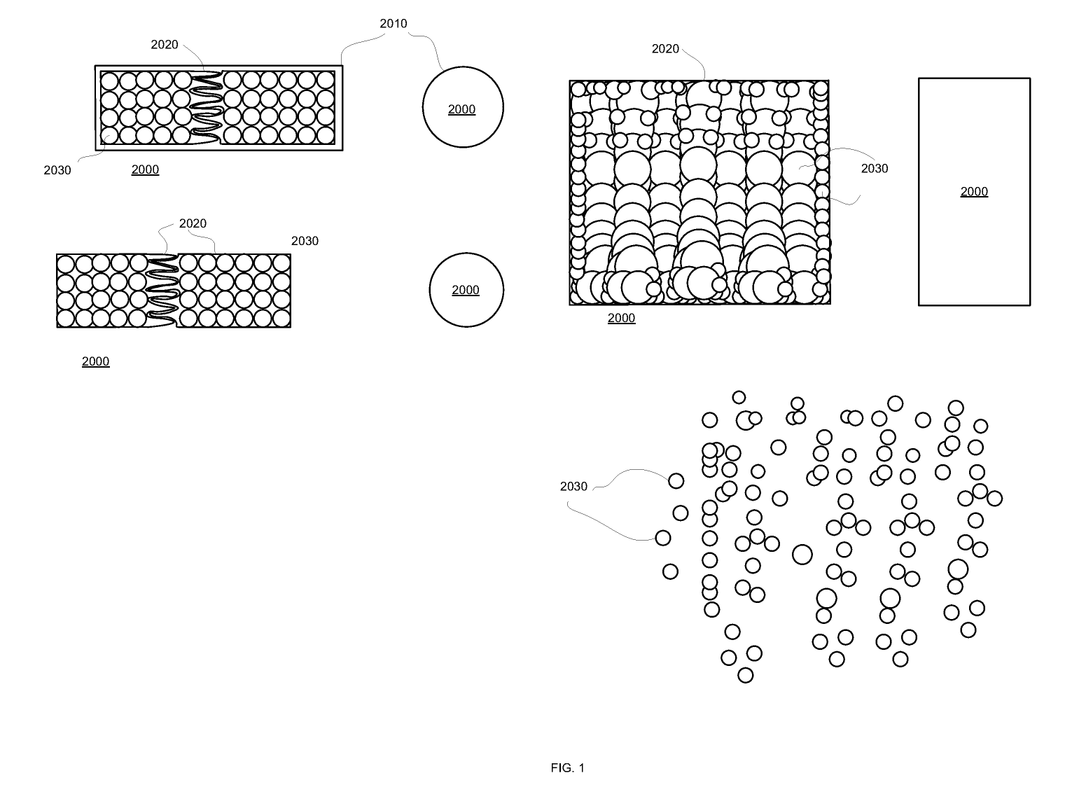

FIG. 1 illustrates expandable polymer elements 2030 that apply force against secondary enclosure 2020 when secondary enclosure 2020 is fully expanded.

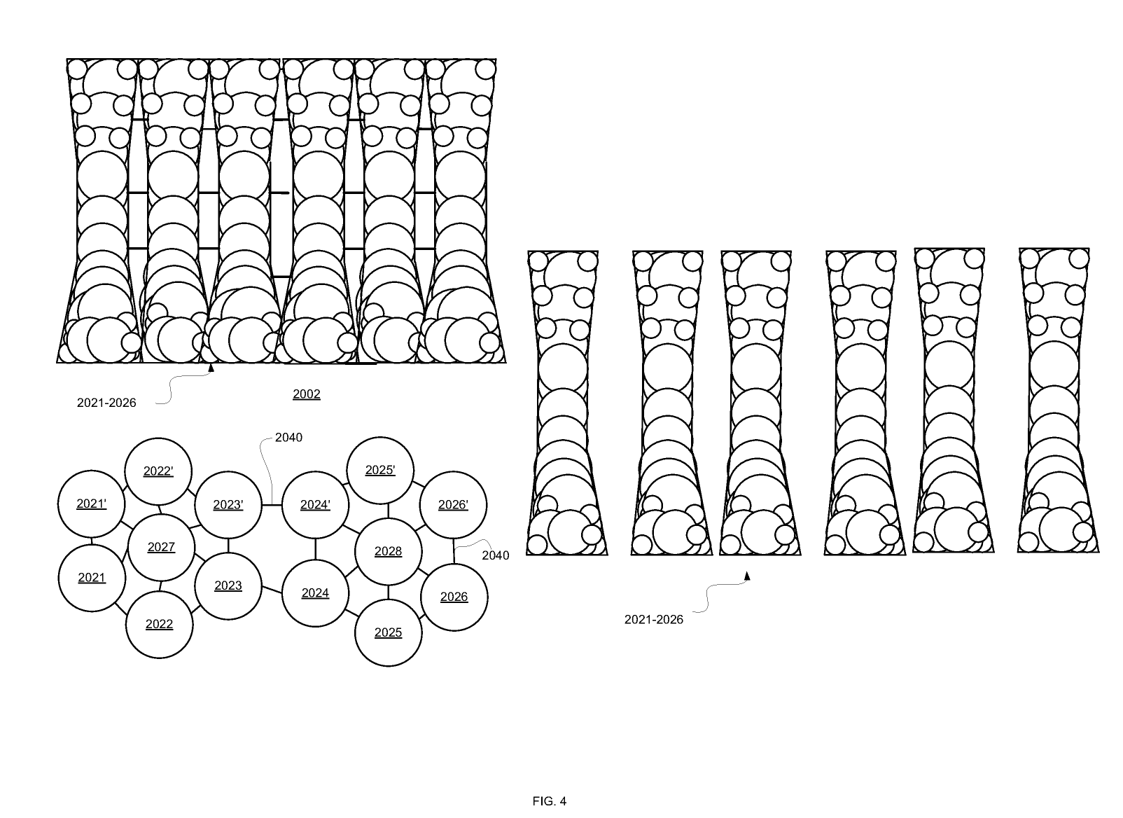

FIG. 4 illustrates expandable polymer elements 2030 that apply force against secondary enclosures 2021, 2021′, 2022, 2022′, 2023, 2023′, 2024, 2024′, 2025, 2025′, 2026, 2026′, 2027 and 2028 when these secondary enclosures are fully expanded.

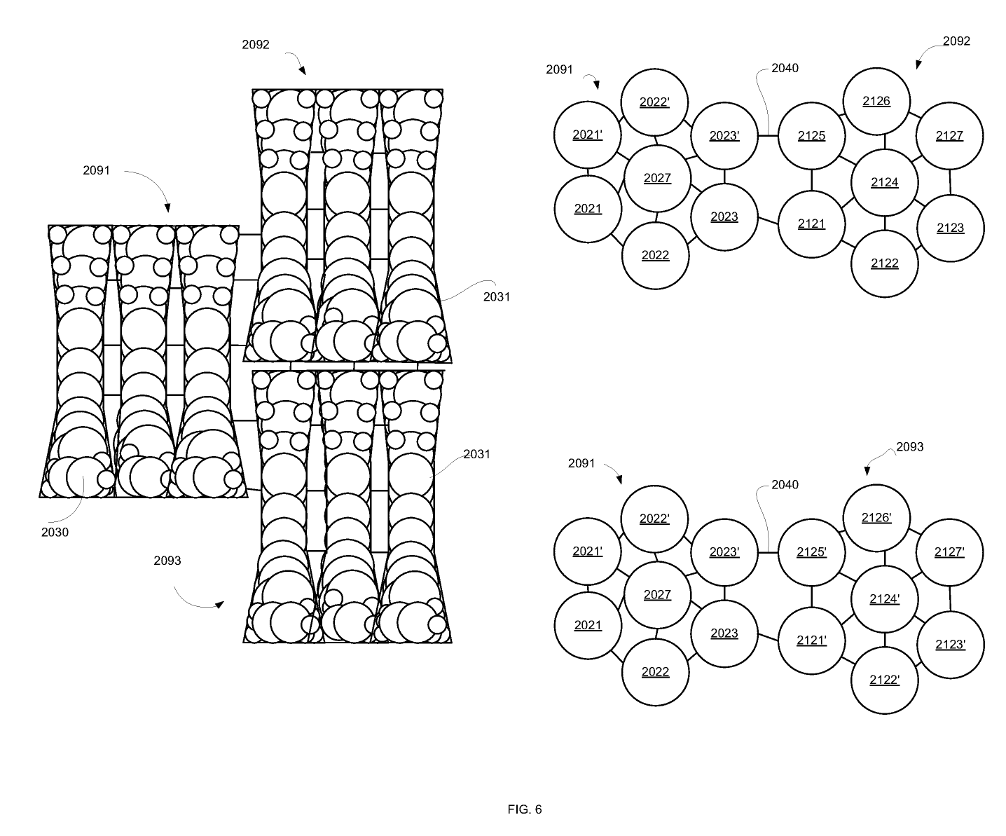

FIG. 6 illustrates expandable polymer elements 2030 that apply force against secondary enclosures 2021, 2021′, 2022, 2022′, 2023, 2023′ and 2027 when these secondary enclosures are fully expanded.

FIG. 6 also illustrates expandable polymer elements 2031 that apply force against secondary enclosures 2121-2127 and 2121′-2127′ when these secondary enclosures are fully expanded.

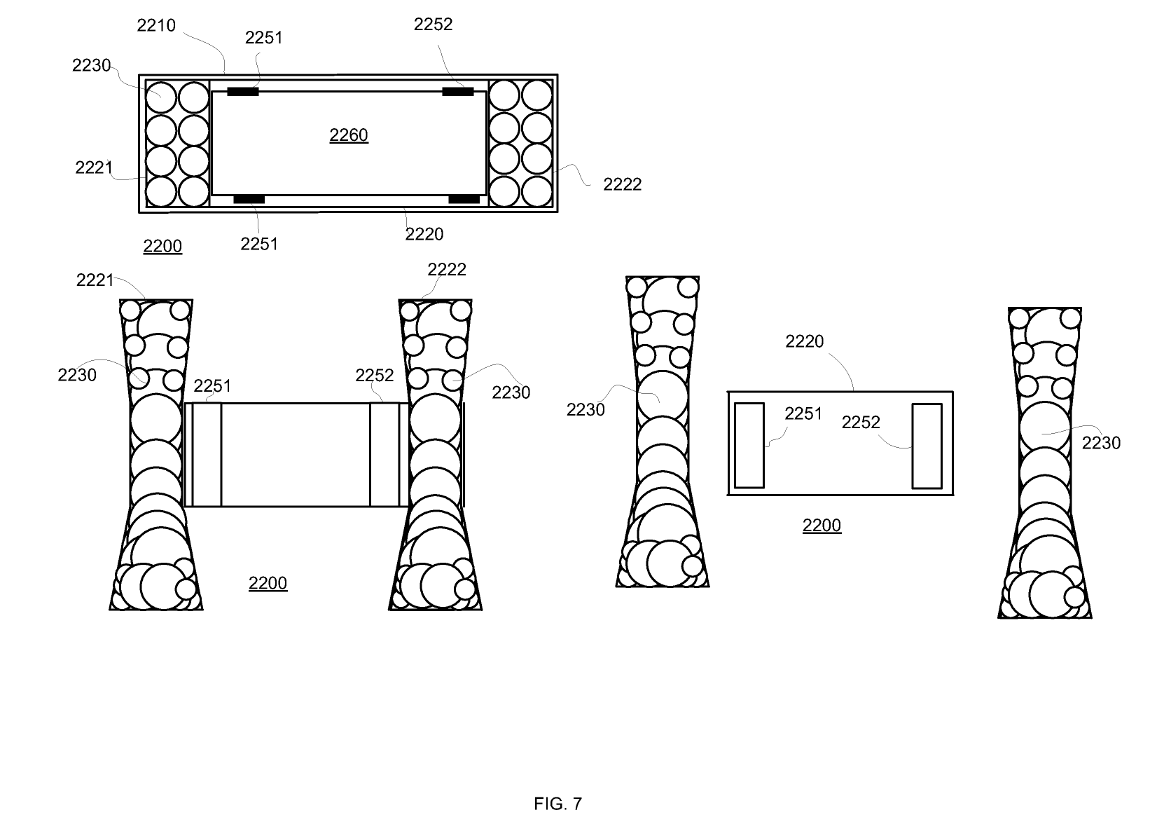

FIGS. 7-8 and 11-13 illustrates expandable polymer elements 2030 that apply force against secondary enclosures 2221 and 2222 when secondary enclosures 2221 and 2222 are fully expanded.

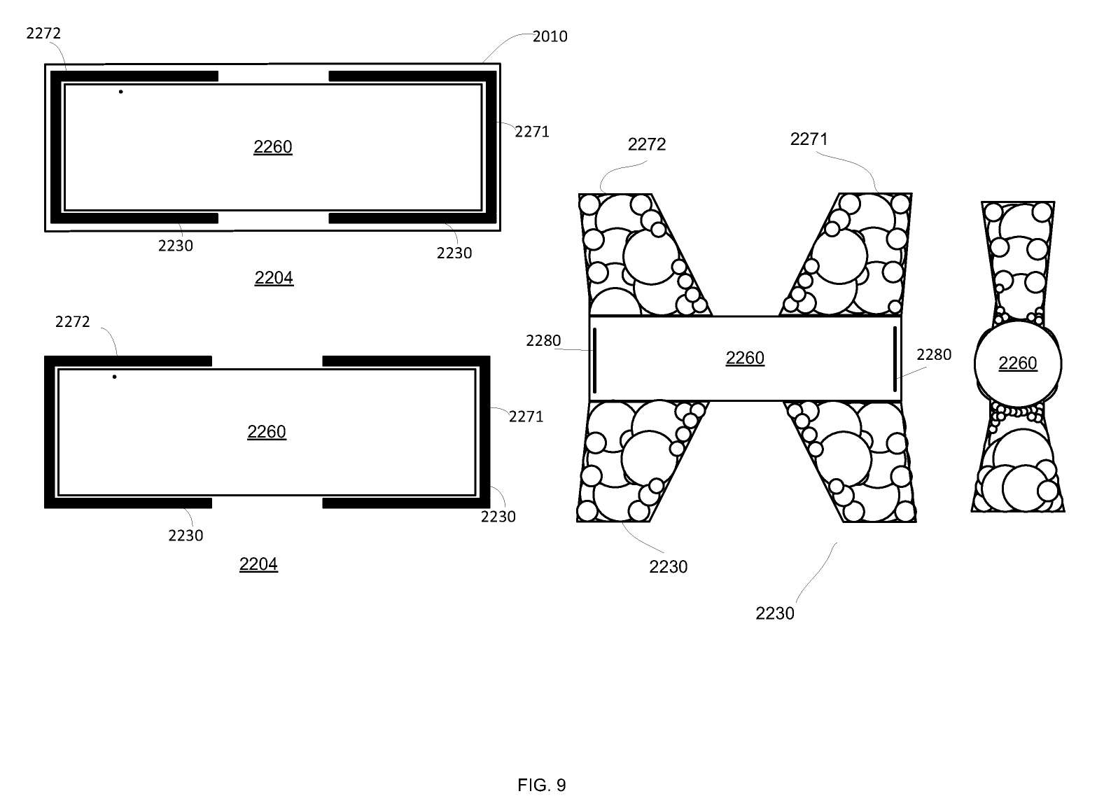

FIG. 9 illustrates expandable polymer elements 2030 that apply force against secondary enclosures 2271 and 2272 when secondary enclosures 2271 and 2272 are fully expanded.

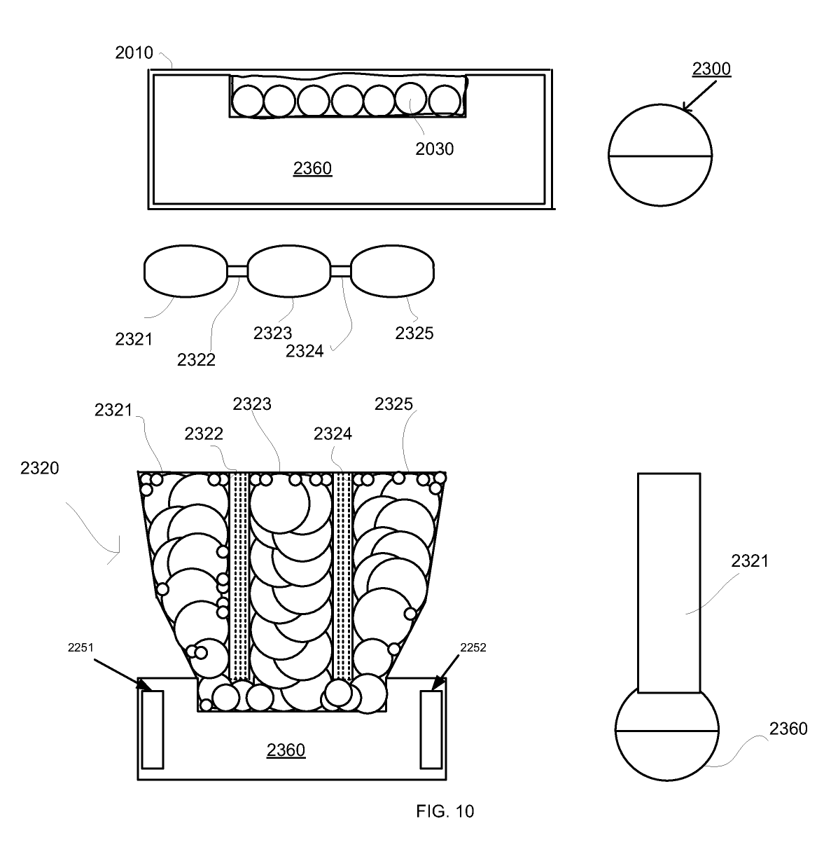

FIG. 10 illustrates expandable polymer elements 2030 that apply force against secondary enclosures 2321, 2323 and 2325 when these secondary enclosures fully expanded.

FIG. 1 illustrates an example of capsule 2000. Capsule 2000 includes outer enclosure 2010, secondary enclosure 2020, and expandable polymer elements 2030.

Capsule 2000 undergoes three stages.

The top left part of FIG. 1 illustrates a first stage of capsule 2000. The first state occurs before the capsule is swallowed by the entity—and may last until outer enclosure 2010 of capsule 2000 is dissolved due to contact with gastric juices.

Secondary enclosure 2020 is within outer enclosure 2010. Expandable polymer elements 2030 are within secondary enclosure 2020.

The bottom left part of FIG. 1 illustrates capsule 2000 at the end of the first stage—outer enclosure 2010 dissolves (due to contact with gastric juices), secondary enclosure 2020 and expandable polymer elements 2030 are in a folded state.

During the second stage the secondary enclosure 2020 fully expands to a fully expanded state and the expandable polymer elements 2030 expand to a partial expanded state—as the secondary enclosure 2020 (even when fully expanded) prevents the expandable polymer elements to reach their fully expanded state.

The fully expanded secondary enclosure 2020 is shown in the upper right part of FIG. 1.

Accordingly—the aggregate volume of the expandable polymer elements 2030 when fully expanded exceeds the volume of the fully expanded secondary enclosure 2020.

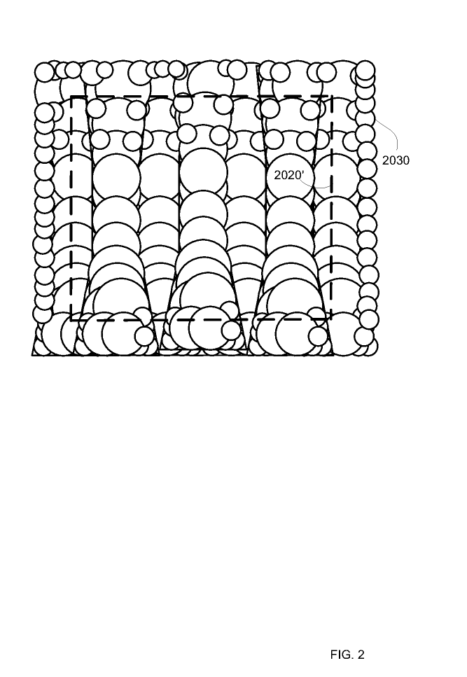

FIG. 2 illustrates an example of e relationship between the fully expanded volume (2020′) of the secondary enclosure and the fully expanded volume of the expandable polymer elements 2030. Any other relationship may be provided.

The fully expanded secondary enclosure 2020 may remain in the stomach until it is partially or fully self-disintegrated.

The third stage starts when the secondary enclosure is partially or fully self-disintegrated and the expandable polymer elements 2030 exit from the stomach. This stage is illustrated in the bottom right part of FIG. 1.

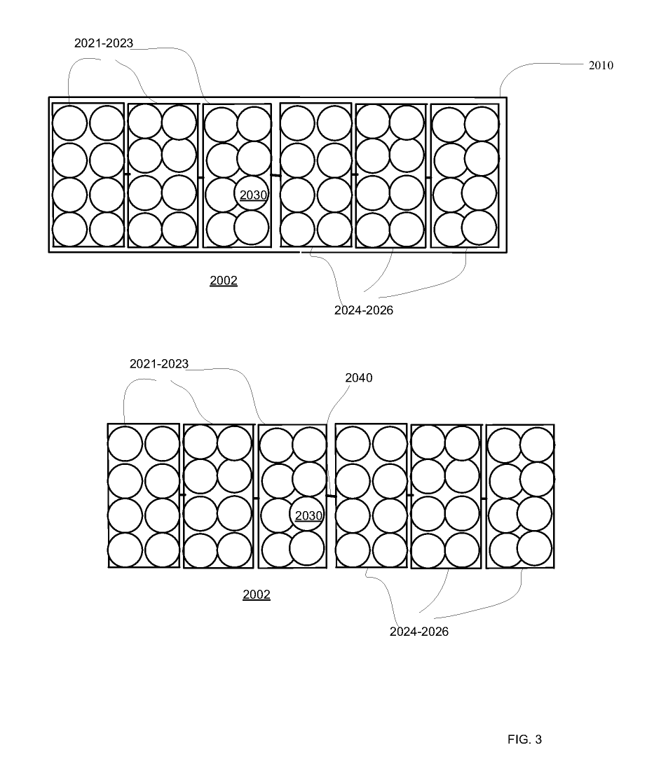

FIGS. 3-4 illustrates an example of capsule 2002. Capsule 2002 includes outer enclosure 2010, secondary enclosures 2021, 2021′, 2022, 2022′, 2023, 2023′, 2024, 2024′, 2025, 2025′, 2026, 2026′, 2027 and 2028, as well as expandable polymer elements 2030.

Capsule 2002 undergoes three stages.

The top part of FIG. 3 illustrates the first stage of capsule 2002. The first stage occurs before the capsule is swallowed by the entity—and may last until outer enclosure 2010 of capsule 2002 is dissolved due to contact with gastric juices.

The secondary enclosures are within outer enclosure 2010. Expandable polymer elements 2030 are within the secondary enclosures. Expandable polymer elements 2030 are within the secondary enclosures.

FIG. 3 is a side view of capsule 2002 that shows only some of the secondary enclosures (2021-2026).

The bottom part of FIG. 3 illustrates capsule 2002 at the end of the first stage—outer enclosure 2010 dissolves (due to contact with gastric juices), secondary enclosures 2021-2026 and expandable polymer elements 2030 are in a folded state.

FIG. 4 illustrates that during the second stage—the secondary enclosures fully expand to a fully expanded state and the expandable polymer elements 2030 expand to a partial expanded state—as the secondary enclosures (even when fully expanded) prevent the expandable polymer elements to reach their fully expanded state.

The fully expanded secondary enclosures 2021, 2021′, 2022, 2022′, 2023, 2023′, 2024, 2024′, 2025, 2025′, 2026, 2026′, 2027 and 2028 are shown in FIG. 4. The top left part of FIG. 4 is a side view and the bottom left part of FIG. 4 is a top view.

The secondary enclosures may remain intact—even after exiting the stomach.

The secondary enclosures are connected to each other by connectors 2040 that are configured to at least partially self-disintegrate after a period of time in the stomach.

The third stage starts when the connectors are dissolved.

Each secondary enclosure (when in fully expanded state) should be shaped and sized to exit the stomach—and may be, for example long and elongated. It may have a length of about 30 mm but a width of about 5 mm.

The right part of FIG. 4 illustrates the secondary enclosures after the connectors 2040 are dissolved.

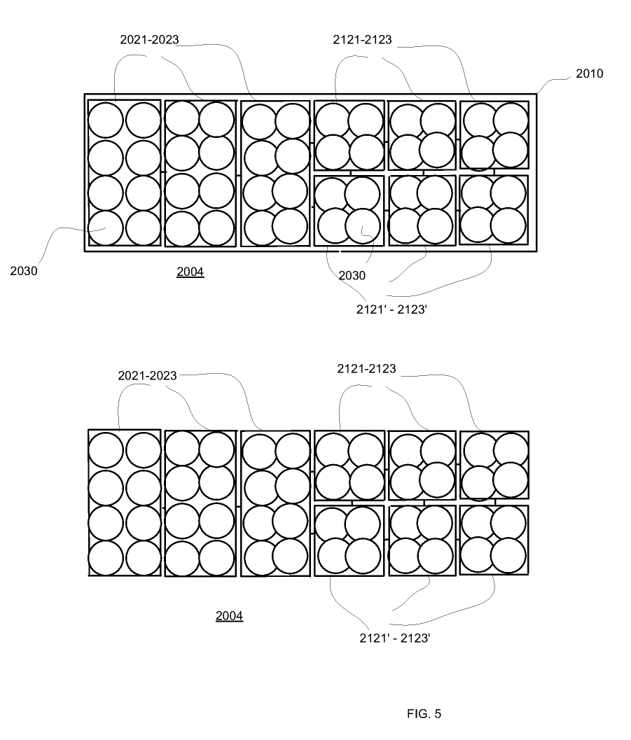

FIGS. 5-6 illustrates an example of capsule 2004. Capsule 2004 differs from capsule 2002 by including more secondary enclosures and different types of expandable polymer elements.

Capsule 2004 includes a first set 2091 of secondary enclosures, a second set 2092 of secondary enclosures and a third set 2093 of secondary enclosures.

First set 2091 of secondary enclosures includes secondary enclosures 2021, 2021′, 2022, 2022′, 2023, 2023′ and 2027. The first set 2901 of secondary enclosures surrounds expandable polymer elements 2030 of a first type.

Second set 2092 of secondary enclosures—that includes secondary enclosures 2121, 2122, 2123, 2124, 2125, 2126 and 2127. The second set 2092 of secondary enclosures surrounds expandable polymer elements 2031 of a second type.

Third set 2093 of secondary enclosures—that includes secondary enclosures 2121′, 2122′, 2123′, 2124′, 2125′, 2126 and 2127′. The third set 2093 of secondary enclosures surrounds expandable polymer elements 2031 of a second type.

Secondary enclosures of the first, second and third sets are connected to each other by connectors 2040 that are configured to at least partially self-disintegrate —thereby disconnecting the secondary enclosures from each other after a period of time in the stomach.

The expandable polymer elements 2030 of the first type may expand faster than the expandable polymer elements 2031 of the second type—in order to prevent the structure to exit the stomach immediately after entering the stomach. The expandable polymer elements 2031 of the second type may have a bigger swelling ratio than the expandable polymer elements 2030 of the first type—in order to occupy a bigger space with the stomach.

Any capsule may or may not include electrical components and/or optical components.

These electrical components and/or optical components may be assembled to form an electronic block—although any other arrangement may be provided.

The electrical components and/or optical components may include at least some out of sensors (radiation sensors, impedance sensors, pH sensors, location sensors, temperature sensors), electrical signal generators, controllers, electrodes, switches, power source, and the like.

Radiation sensors may include light sensors, radars, sonars, infrared sensors, and the like.

Impedance sensors, pH sensors and radiation sensors may be used as location sensors for determining the location of the capsule in the subject.

Electrodes may be part of a location sensor and/or may be used to output electrical signals—for example—for stimulating the vagus nerve.

Various examples of electrical components and/or optical components that can be included in the capsule are illustrated in PCT patent application PCT/IL2018/050043 which is incorporated herein by reference in its entirety.

Each of the capsules mentioned in the specification and/or drawings can include none of these electrical components and/or optical components or at least one of these electrical components and/or optical components.

FIG. 7 illustrates a capsule 2200 that includes a housing 2260 that may (or may not) be hermetically sealed. One or more of the electrical components and/or optical components may be surrounded by the housing. A sensor or another component may extend outside the housing in order to sense the surroundings of the housing.

In FIG. 7 electrodes 2251 and 2252 extend outside the housing.

FIG. 7 illustrates two secondary enclosures 2221 and 2222 that are connected to housing 2260 by dissolvable connectors (not shown).

Expandable polymer elements 2030 are positioned in a right space defined between secondary enclosure 2222 and a right sidewall of housing 2260.

Expandable polymer elements 2030 are positioned in a left space defined between secondary enclosure 2221 and a left sidewall of housing 2260.

The top left part of FIG. 7 illustrates a first stage of capsule 2200. Outer enclosure 2010 surrounds housing 2260 and secondary enclosures 2221 and 2222. Secondary enclosures 2221 and 2222 as well as and expandable polymer elements 2030 are in a folded state.

During the second stage the secondary enclosures 2221 and 2222 fully expands to fully expanded state and the expandable polymer elements 2030 expand to a partial expanded state—as the secondary enclosures 2221 and 2222 (even when fully expanded) prevent the expandable polymer elements to reach their fully expanded state.

The fully expanded secondary enclosures 2221 and 2222 are shown in the bottom left part of FIG. 7.

During the third stage the secondary enclosures 2221 and 2222 detach from the housing 2260 due to the dissolving of the connectors—and each individually exits the stomach. See the right side of FIG. 7.

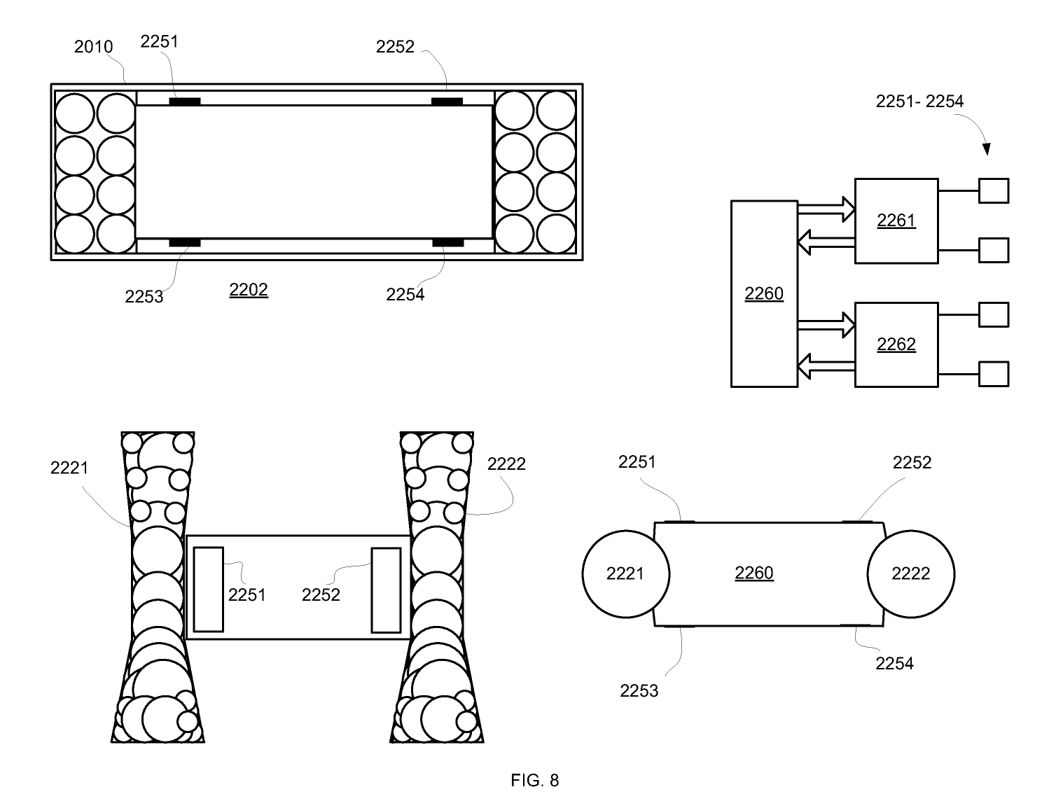

FIG. 8 illustrates capsule 2202. Capsule 2202 differs from capsule 2200 by having four electrodes—2251, 2252, 2253 and 2254. First and second electrodes 2251 and 2252 extend from one side of housing 2260—while third and fourth electrodes 2253 and 2254 extend from another side of housing 2260.

After determining the location of the capsule—and especially which side of the capsule contacts the stomach wall—the capsule may select to activate only the electrodes that are in contact with the stomach—thereby saving energy and providing a more efficient stimulation.

The top right part of FIG. 8 illustrates a controller 2260 that determines which pair of electrodes to activate—and instructs either first generator 2261 to send pulses through electrodes 2251 and 2252—or instructs second 2262 to send pulses through electrodes 2253 and 2254.

FIG. 9 illustrates a capsule 2204 that includes a housing 2260 that may (or may not) be hermetically sealed. One or more of the electrical components and/or optical components may be surrounded by the housing. A sensor or another component may extend outside the housing in order to sense the surroundings of the housing.

In FIG. 9 electrodes 2280 extend outside the housing.

FIG. 9 illustrates two secondary enclosures 2271 and 2272 that are connected to housing 2260 by dissolvable connectors (not shown).

Expandable polymer elements 2030 are positioned in a right space defined between secondary enclosure 2271 and a right portion of an exterior of housing 2260. The right portion is illustrated as including a right sidewall, a right part of the bottom of the housing and a right part of the top of the housing.

Expandable polymer elements 2030 are positioned in a left space defined between secondary enclosure 2271 and a left portion of an exterior of housing 2260. The left portion is illustrated as including a left sidewall, a left part of the bottom of the housing and a left part of the top of the housing.

The top left part of FIG. 9 illustrates a first stage of capsule 2204.

The bottom left part of FIG. 9 illustrates capsule 2204 at the end of the first stage—outer enclosure 2010 dissolves (due to contact with gastric juices), secondary enclosures 2271 and 2272 as well as and expandable polymer elements 2030 are in a folded state.

During the second stage the secondary enclosures 2271 and 2272 fully expands to fully expanded state and the expandable polymer elements 2030 expand to a partial expanded state—as the secondary enclosures 2271 and 2272 (even when fully expanded) prevent the expandable polymer elements to reach their fully expanded state.

The fully expanded secondary enclosures 2271 and 2272 are shown in the right part of FIG. 9.

The third stage is not shown. During the third stage the secondary enclosures 2271 and 2272 detach from the housing 2260 due to the dissolving of the connectors—and each individually exits the stomach.

FIG. 10 illustrates capsule 2300 that includes a set of secondary enclosures 2321, 2323 and 2325 that surround expandable polymer elements 2030 and extend (after outer enclosure 2010 dissolves) from a cavity formed in a top of housing 2360. These secondary enclosures are formed in a row and are spaced apart from each other by bridging elements 2322 and 2324.

FIG. 10 also illustrates electrodes 2251 and 2252 that extend from housing 2360.

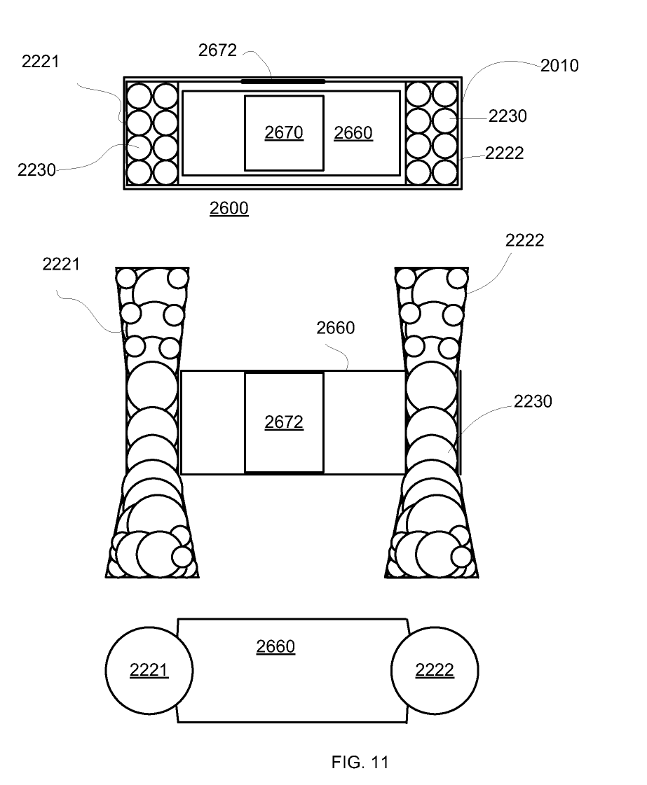

FIG. 11 illustrates capsule 2600 that includes outer enclosure 2010, secondary enclosures 2221 and 2222, expandable polymer elements 2030, housing 2660 that includes a window 2672 and an optical elements 2670 such as camera or light source that may transmit and/or receive light through window 2672.

Expandable polymer elements 2030 are positioned in a right space defined between secondary enclosure 2222 and a right sidewall of housing 2260. Expandable polymer elements 2030 are positioned in a left space defined between secondary enclosure 2221 and a left sidewall of housing 2260.

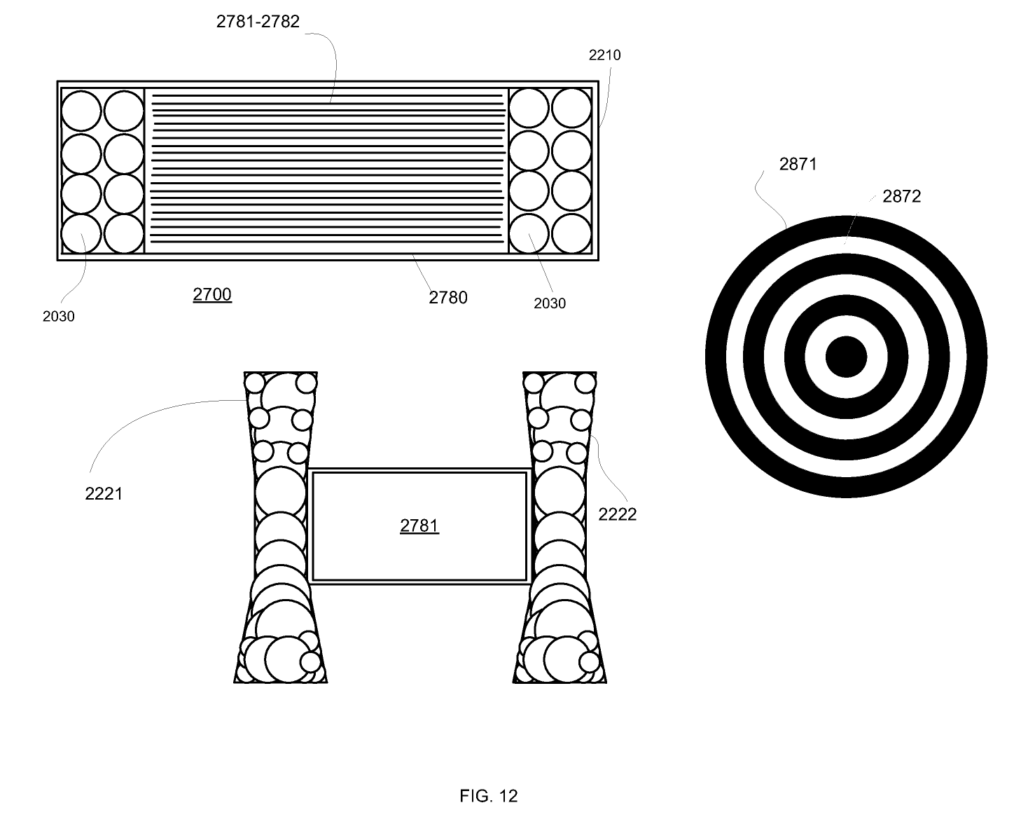

FIG. 12 illustrates capsule 2700 that has a gradual/slow drug release system—interleaved coaxial layers of protective coating (for example glucose) 2871 and drug 2872 are provided. These layers are gradually disintegrated when exposed to gastric juice—thereby exposing one drug layer after the other. Different drug layers may include the same drug or may include different drugs. Different layers may be of different thickness and/or different dissolving rate.

The interleaved coaxial layers are positioned in housing 2780. Expandable polymer elements 2030 are positioned in a right space defined between secondary enclosure 2222 and a right sidewall of housing 2780. Expandable polymer elements 2030 are positioned in a left space defined between secondary enclosure 2221 and a left sidewall of housing 2780.

The top left part of FIG. 12 is a cross sectional view of the capsule 2700 when the outer enclosure 2210 is intact and secondary enclosures 2221 and 2222 are in a folded state.

The bottom left part of FIG. 12 is a side view of the capsule 2700 after the outer enclosure 2210 dissolves and secondary enclosures 2221 and 2222 are fully expanded.

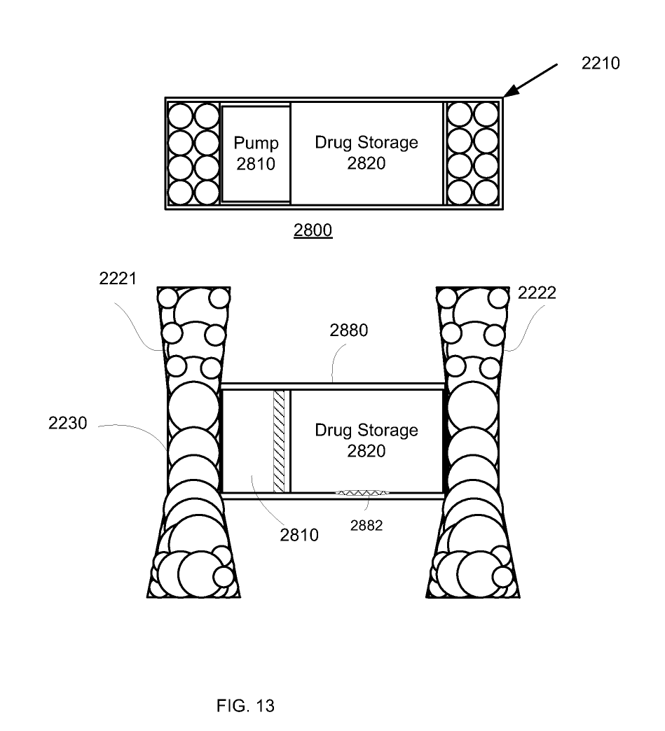

FIG. 13 illustrates capsule 2800 that has a drug storage 2820 for storing a drug and a drug delivery system (DDS) such as a drug release system—that may be a pump 2810. Drug storage 2820 and pump 2810 are stored within housing 2880 that has an opening/membrane 2882 for outputting drug released by pump 2810. The opening may be permanently or only temporarily open.

Expandable polymer elements 2030 are positioned in a right space defined between secondary enclosure 2222 and a right sidewall of housing 2880. Expandable polymer elements 2030 are positioned in a left space defined between secondary enclosure 2221 and a left sidewall of housing 2880.

The top part of FIG. 13 is a cross sectional view of the capsule 2800 when the outer enclosure 2210 is intact and secondary enclosures 2221 and 2222 are in a folded state.

The bottom part of FIG. 13 is a side view of the capsule 2800 after the outer enclosure 2210 dissolves and secondary enclosures 2221 and 2222 are fully expanded.

It may be beneficial to deactivate the internal power supply of the capsule after assembly and to activate it only when needed—especially when the capsule may be consumed weeks and even months after it is manufactured and stored.

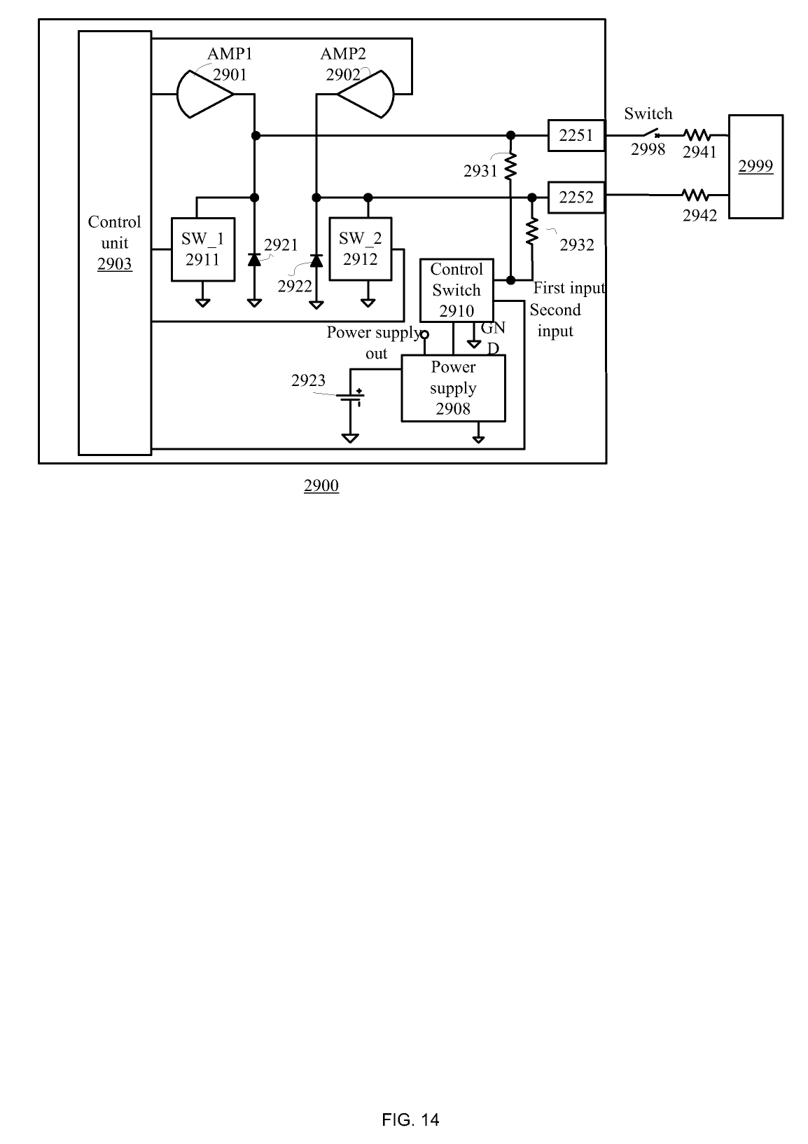

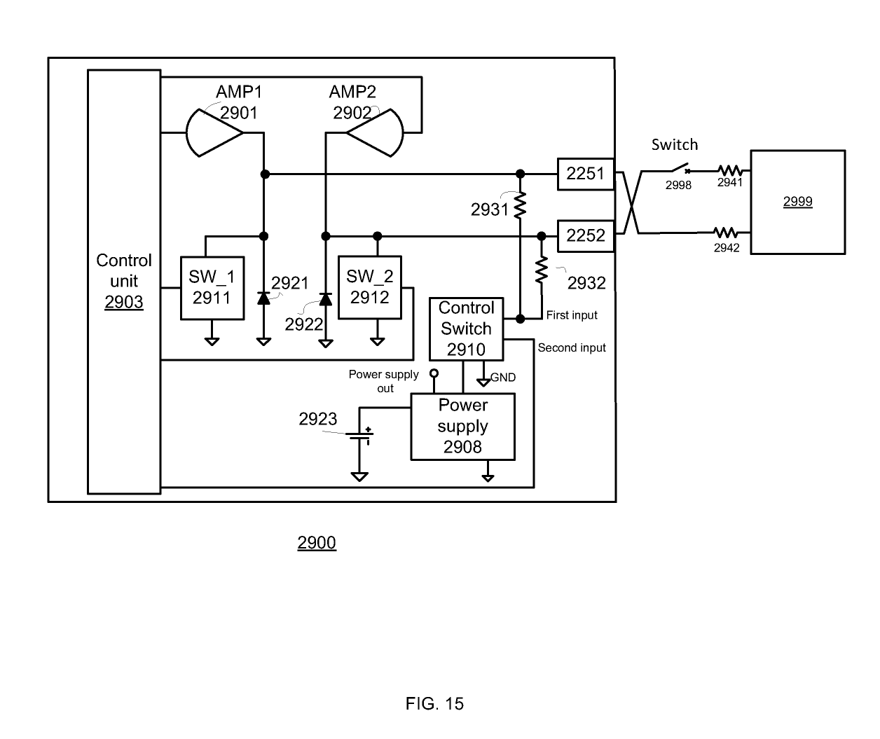

FIGS. 14 and 15 illustrate capsule 2900 and a capsule activator.

The capsule activator includes an external power supply unit 2999, switch 2998 and resistors 2941 and 2942.

Capsule power supply 2908 is switched “off” before use and during storage/shelf life.

FIG. 14 illustrates that a positive pole of 2999 is coupled (via resistor 2941 and switch 2998) to electrode 2251.

When switch 2998 is closed (short connection), electrical voltage from external power supply 2999 is applied to the first input of control switch 2910 via resistor 2941, switch 2998, electrode 2251 and resistor 2931. Then, via common wire (GND), diode 2922, electrode 2252 and resistor 2942 voltage closes on return output of 2999.

Control switch 2910 turns “on” and turns “on” power supply 2908. Power supply 2908 supplies voltage to all electronic components of the capsule.

The control unit 2903 turns “on” and sets on the second input of 2910 a signal that allows to hold power supply 2908 in the “on” state after the switch 2998 returns to its disconnected position.

In FIG. 15 the positive pole of external power supply 2999 is connected to electrode 2252. In this option the control voltage is connected to the first input of control switch 2910 via electrode 2252 and resistor 2932 and returns back to external power supply 2999 via common wire (GND), diode 2921 and electrode 2251.

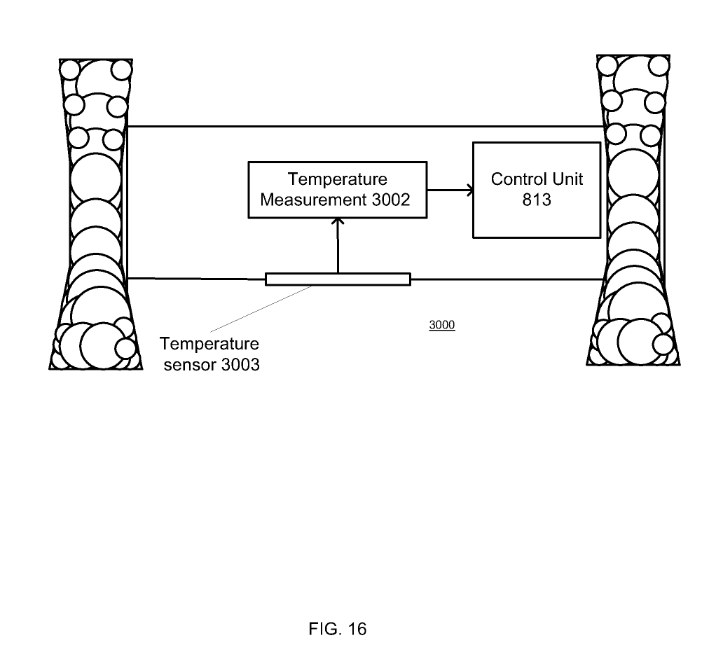

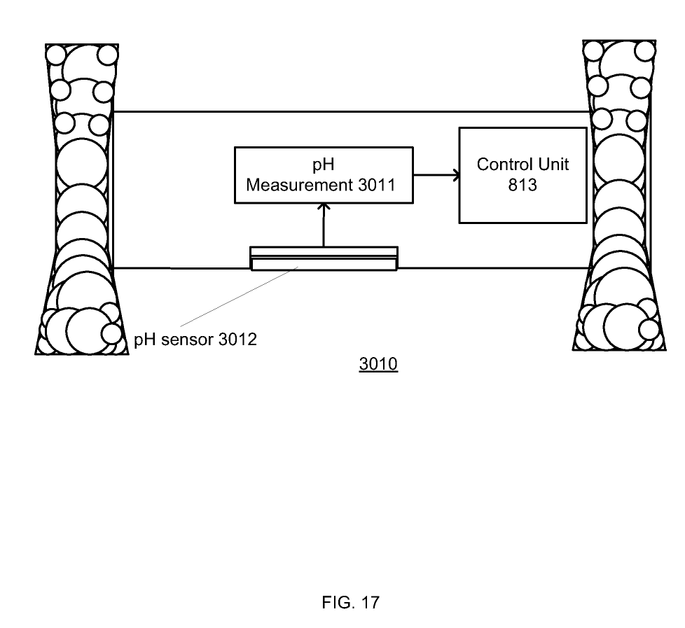

FIGS. 16 and 17 illustrates capsules 3000 and 3010 that include various sensors.

FIG. 16 illustrates capsule 3000 that has a temperature sensor 3003, a temperature measurement unit 3002 and a control unit 813.

FIG. 17 illustrates capsule 3010 that has a pH sensor 3012, a pH measurement unit 3011 and a control unit 813.

Any of these sensors and measurement units may be includes in at least some of the capsules of the previous figures.

The phrase “may be X” indicates that condition X may be fulfilled. This phrase also suggests that condition X may not be fulfilled.

The terms “including”, “comprising”, “having”, “consisting” and “consisting essentially of” are used in an interchangeable manner. For example—any method may include at least the steps included in the figures and/or in the specification, only the steps included in the figures and/or the specification.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

Any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one as or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements the mere fact that certain measures are recited in mutually different claims does not indicate that a combination of these measures cannot be used to advantage.

Any combination of any component of any capsule that is illustrated in any of the figures and/or specification and/or the claims may be provided.

Any combination of steps, operations and/or methods illustrated in any of the figures and/or specification and/or the claims may be provided.

1. A capsule to be ingested by a subject, the capsule comprises:
an outer enclosure configured to dissolve in gastric juice;
a secondary enclosure that is permeable for gastric juice, is at least partially surrounded by the outer enclosure when in a folded state, and has a fully expanded volume; and
an expandable polymer element that is surrounded by the secondary enclosure;
wherein a fully expanded volume of the expandable polymer element exceeds the fully expanded volume of the secondary enclosure.

2. The capsule of claim 1 wherein the secondary enclosure is configured to at least partially self-disintegrate after a first period of time in gastric juice.

3. The capsule of claim 1 wherein the secondary enclosure is configured to withstand gastric juice until exiting the stomach.

4. The capsule of claim 1 wherein the expandable polymer element is configured to be formed from at least one out of dry granules, pellets, powders, micro tablets, mini tablets, and compressed element.

5. The capsule of claim 4 wherein the secondary enclosure comprises multiple apertures with shapes and sizes that prevents exit of the at least one of the dry granules, pellets, powders, micro tablets, mini tablets, and compressed element.

6. The capsule of claim 1 wherein the secondary enclosure is hydrophilic.

7. The capsule of claim 1 wherein the secondary enclosure comprises at least two sections.

8. The capsule of claim 1, wherein the secondary enclosure, when in an expanded state, is configured to be stuck in an antrum region of the subject until an expiration of the first period of time.

9. The capsule of claim 1, comprising secondary enclosures that are at least partially surrounded by the outer enclosure when in a folded state.

10. The capsule according to claim 9, wherein the secondary enclosures are connected to each other by at least one connector that is configured to at least partially self-disintegrate after a second period of time in gastric juice.

11. The capsule of claim 10, wherein the secondary enclosures, while in an expanded state and while connected to each other, form a structure that is stuck in the stomach.

12. The capsule of claim 10, wherein the secondary enclosures, while in an expanded state and after disconnecting from each other, are small enough to individually exit through the pylorus of the subject.

13. The capsule of claim 10, wherein a first secondary enclosure surrounds a first set of expandable polymer elements having a first swelling ratio and a first expansion profile; and wherein a second enclosure surrounds a second set of expandable polymer elements having a second swelling ratio and a second expansion profile; and wherein the first swelling ratio is smaller than the second swelling ratio.

14. The capsule of claim 13, wherein the first expansion profile exhibits a higher expansion rate than the second expansion profile.

15. The capsule of claim 1, comprising a housing that surrounds an electronic block.

16. The capsule of claim 15, wherein the housing comprises electrodes, and wherein the electronic block comprises a power source, a control unit with a wireless communication module and a generator.

17. The capsule of claim 15, comprising a temperature sensor

18. The capsule of claim 15, wherein the housing is hermetically sealed.

19. The capsule of claim 15, wherein the housing is partially transparent.

20. The capsule of claim 19, comprising a camera that is configured to obtain images through the housing.

21. The capsule of claim 19, comprising a light source.

22. The capsule of claim 15, comprising a pH sensor.

23. The capsule of claim 1, comprising a drug storage and a third secondary enclosure that surrounds the drug storage.

24. The capsule of claim 23, wherein the drug storage stores a drug in a slow—release packaging having a pre-determined period of self-disintegration.

25. The capsule of claim 23, wherein the third secondary enclosure comprises an electronic block.

26. The capsule of claim 23, wherein the electronic block comprises a power source, a control unit and a drug delivery system.

27. The capsule of claim 26, wherein drug delivery system is a drug delivery pump.

28. The capsule according to claim 1 comprising a control switch and an internal power supply unit; wherein the control switch, once fed with a control signal from a capsule activator, is configured to activate the internal power supply unit.

29. The capsule according to claim 28 wherein the control switch is coupled to electrodes of the capsule.

30. The capsule according to claim 1 comprising a control unit, a first electrode, a second electrode, a control switch that has a first input and a second input, and an internal power supply; wherein the first input is coupled to the first electrode; wherein the second input is coupled to the control unit; wherein the control switch is coupled to the internal power supply; wherein the control switch, once fed with a voltage from an external power supply is configured to activate the internal power supply.
